(19) United States
(12) Patent Application Publication
(10) Pub. No.: US 2022/0340851 A1
(43) Pub. Date: Oct. 27, 2022

(54) MELT ELECTROWRITTEN FILTER FOR CAPTURING CELLS

(71) Applicant: Aarhus Universitet, Aarhus C (DK)

(72) Inventors: Menglin Chen, Aarhus C (DK); Mathias Lindh Jørgensen, Randers SØ (DK); Mille Sikkersoq Kimer Nøddesbo, Risskov (DK)

(21) Appl. No.: 17/753,025

(22) PCT Filed: Aug. 28, 2020
(86) PCT No.: PCT/EP2020/074041

§ 371 Date: Feb. 15, 2022

(30) Foreign Application Priority Data
Aug. 29, 2019 (EP) 19194329.9

(51) Int. Cl. C12M 1/12; C12M 1/00; C12N 5/09; D01D 5/00

(52) U.S. Cl. CPC C12M 25/02; C12M 47/04; C12N 5/0693; D01D 5/0076; D01D 5/0023; D01F 6/625

(57) ABSTRACT

The present invention relates to thin filters comprising melt electro spinning writing (MEW) fibers for capturing and culturing circulating tumor cells (CTCs). The invention further relates to processes for producing the filters, methods for capturing and culturing CTCs using the filters, kits and devices comprising the filters and uses of the filters.

### TECHNICAL FIELD OF THE INVENTION

The present invention relates to a filter produced using melt electrospinning writing (MEW). The filter enables for affinity based capture of cells. In particular, the present invention relates to a filter for capturing and culturing circulating tumour cells (CTCs) in a fluid sample.

### BACKGROUND OF THE INVENTION

At present, late stage colorectal cancer is associated with poor survival rates, and colorectal cancer is one of the leading causes of cancer related deaths. When colorectal tumors are diagnosed at late stage, there are often limited options in terms of curative surgery and chemotherapy. The tumor heterogeneity leads to complexity, and it can thus be challenging to predict the optimal treatment where significant patient-to-patient differences in treatment response is a pivotal challenge. Circulating tumor cells (CTCs) might be one of the avenues to overcome these challenges. Several studies have shown that stable CTC cell lines can be established as cultures, and it has been suggested that this will aid the understanding of CTC biology and the mechanisms underlying dissemination of cancer. However, it can be difficult to establish CTC derived cell lines of colorectal cancer patients because of the low number of CTCs in blood of these patients.

Recently, there has been an increasing interest in CTC-derived tumor material for precision medicine development and drug testing platforms.

Electrospinning is a robust nanofiber-producing technique, where viscous liquids made of virtually any polymers, supramolecules or composites can be extruded under an electric field and elongated as submicron fibers. Nano- and submicron fibers have an enormous variety of applications, which is rooted in their ultrahigh surface area. Extensive modifications of the fibers have been achieved, and controlling fiber positioning and alignment can be manipulated by changing the electric field strength and by utilizing dynamic collectors. Among electrospinning techniques, melt electrospinning writing (MEW) is distinguished by the possibility to produce highly defined architectures in the lower micrometer to submicrometer range.

Zhang et al. discloses an electrospun TiO2 nanofiber-based cell capture assay for detecting circulating tumor cells from colorectal and gastric cancer patients (Adv Mater. 2012 May 22; 24(20): 2756-60). In Zhang et al the material is spun onto a surface and does not relate to filters. Further, Zhang is silent in respect of culturing CTCs on the surface.

Yu et al. discloses an electrospinning process to prepare poly(lactic-co-glycolic acid) (PLGA) nanofibrous arrays in random or aligned orientations on glass slips (J Nanobiotechnol (2019) 17: 31). In Yu et al the material is spun onto a surface and does not relate to filters. Further, Yu is silent in respect of culturing CTCs on the surface, instead CTCs are released from the surface.

Takayuki U et al discloses a three-dimensional polystyrene (PS) microfiber fabric filter with vacuum aspiration system developed for capturing circulating tumor cells (CTCs) within a short time (SCIENCE AND TECHNOLOGY OF ADVANCED MATERIALS, vol. 17, no. 1, 25 Nov. 2016, pages 807-815.

Delalat B et al discloses a 3D lattice manufactured by melt electrospinning writing (MEW) PCL. The lattices were coated via plasma polymerization to introduce reactive functional groups for covalent anti-CD3 and anti-CD28 antibody attachment in a conformal coating around the fibers (PCL) of the 3D lattice (BIOMATERIALS, vol. 140, 7 Jun. 2017, pages 58-68).

Gagandeep K et al discloses electrospun fiber mats (polycaprolactone) with immobilized capture agents for culturing cells (ACS OMEGA, vol. 4, no. 2, 27 Feb. 2019, pages 4376-4383).

Thomas M. R. et al reviews various forms and applications of melt electrowriting including the preparation of PCL microfiber meshes coated with antibodies to enhance cell adherence (ADVANCED FUNCTIONAL MATERIALS, vol. 29, no. 44, 18 Aug. 2019, page 1904664 (1-28).

Hence, an improved method for capturing CTCs would be advantageous, and in particular a more efficient and/or reliable method for culturing CTCs would be advantageous.

### SUMMARY OF THE INVENTION

Herein, a melt electrospinning writing (MEW) polycaprolactone (PCL) filter for capturing and (in vitro) culturing of CTCs is presented (examples 1 and 2). This PCL filter allows for culturing and for subsequent down-stream analysis such as flow cytometry, drug resistance tests, immunocytochemistry and western blotting. In the presented work, the HT29 colon cancer cell line has been used to show that colon cancer cells can be captured in a background of whole blood (example 5). It is also demonstrated that the HT29 cells can be grown on the filters at clinical relevant seeding densities (example 4), and that colon cancer cells can be filtrated and subsequently expanded/cultured directly on the filters, also at clinically relevant concentrations (example 4). Further, FIG. 1C and 1D show produced filters according to the invention and FIG. 1F schematically shows the filter (1) and the layers (4, 5). Further, the apparent 2D structure of the filter and its transparent nature (FIG. 1C), makes it possible to directly analyze the filter under the microscope.

Thus, an object of the present invention relates to the provision of a novel filter for capturing cells, especially CTCs.

In particular, it is an object of the present invention to provide a novel capturing method for CTCs that solves the above mentioned problems of the prior art with capturing and culturing of CTCs, without having to removing cells from filters before culturing.

It is also an object of the present invention to provide a filter which can be directly visualized using a light microscope.

Thus, one aspect of the invention relates to a filter (1) comprising
a frame layer (4) of fibers (2); preferably comprising cell capturing moieties (3), preferably antibodies;
a filtering layer (5) of fibers (2), comprising cell capturing moieties (3), preferably antibodies; and

wherein the filtering layer (5) has a thickness in the range 1-100 μm.

In a preferred embodiment, the fibers (2) are fabricated by melt electrospinning writing (MEW), preferably PCL melt electrospinning writing (MEW) fibers. In another preferred embodiment, the filter is transparent and/or has a 2D shape.

Another aspect of the present invention relates to a method for capturing cells, preferably circulating tumor cells (CTCs), the method comprising
a) providing a filter (1) according to the invention;
b) passing a sample suspected of comprising cells through the filter (1), thereby capturing the cells bound by the cell capturing moieties (3).

Yet another aspect of the present invention is to provide a method for capturing and culturing cells, preferably CTCs, the method comprising
a) performing the method (for capturing cells) according to the invention;
b) transferring the filter (suspected of comprising captured cells, preferably CTCs) to a culturing medium for the cells; and
c) culturing the cells on the filter, such as for at least 1 day in the medium.

Still another aspect of the present invention is to provide a process for producing a filter (1) according to the invention, the process comprising
melt electrospinning writting (MEW) a filtering layer (5) on a surface;
melt electrospinning writting (MEW) a frame layer (4) on the filtering layer (5);
coating at least the filtering layer (5) (preferably both layers) with cell capturing moieties (3); and
providing a filter (1) according to the invention.

An additional aspect relates to a filter (1) obtained/obtainable by the process (of producing a filter) according to the invention.

Yet an additional aspect relates to a filtering device, such as a filter holder comprising a filter (1) according to any the invention.

An aspect also relates to a cell culturing device comprising a filter (1) according to the invention.

Yet another aspect relates to a microscope comprising a filter (1) according to any the invention, positioned for microscopy analysis.

An aspect relates to a kit of parts comprising
a filter (1) according to the invention;
a filter holder for the filter;
optionally, buffers for anticoagulation;
optionally, one or more syringes; and
optionally, instruction for use.

An aspect also relates to the use of a filter (1) according to the invention, for filtering a sample, such as a sample selected from a biological sample, preferably a blood sample, such as whole blood, blood serum or blood plasma, urine, or Cerebrospinal fluid (CSF).

A final aspect relates the use of a filter according to the invention, for (in vitro) capturing and optionally culturing cells, preferably CTCs.

### BRIEF DESCRIPTION OF THE FIGURES

FIG. 1

Schematic illustration of the used electrospinning techniques: A) illustration of solution electrospinning. PCL was dissolved in either CF or DMF/DCM and electrospun onto a solid surface. B) The melt electrospinning writing method, which was the preferred method for producing the invention. PCL polymer is melted inside the needle and forced down onto a moving plate. The plate moves according to a software programme that uses G-code. Hereby can the pattern be controlled. The speed by which the plate moves can also be controlled. Fast speed creates linear fibres whereas coiled fibres are created at low speeds. The filter and the setup: C) The filter is transparent, scale bar 1.5 cm. D) An electron microscope image of the filter, scalebar 300 um. The frame layer (4) is the linear thick fibres whereas the coiled fibres create the filtering layer (5). E) The filtration setup, where the filter is mounted onto a filter holder, the holder is then attached to a syringe onto which the blood sample is loaded. The syringe and the filter holder is connected to a syringe pump. The pump controls the flow rate. F) Schematic illustration of the filters of the invention. G) Schematic illustration of a filtering setup using the filter of the invention. H) Schematic illustration of a filtering process according to the invention. I) Schematic illustration of the filter placed in a culturing medium (top) and picture of cultured cells on the filter (bottom).

FIG. 2

The three filter types, electron microscope images: A) The DCM filter, A1) the DCM filter image skeletonised in ImageJ, used for filter characterisation. B) The CF filter, B1) the CF filter image skeletonised in ImageJ, used for filter characterisation. C) The MEW filter, C1) the MEW filter image skeletonised in ImageJ, used for filter characterisation. Scalebar, 100 μm.

FIG. 3

Filtration of 2000 HT29 colon cancer cells: A) Image of a filter not conjugated to an antibody. No bright dots can be seen, which means that no cells were captured. B) A filter with conjugated anti EpCAM antibody on the surface. Here, there are many bright dots; many cancer cells were captured.

FIG. 4

Culture of a few HT29 cancer cells and downstream analysis: A) CCK-8 assay for cell viability measurement. After 15 days the cells had expanded well for all seeding densities, 100, 200, and 400 cells per filter. B) Cells on a “100 cell seeding” filter was visualised by fluorescence microscopy, scalebar 40 μm. C) Flow cytometry: Cultured cells were released from the filter. It was shown by flow cytometry that they express EpCAM on their surface. Western blot: D) The presence of EpCAM was further confirmed by western blotting and the presence of beta actin was also shown by western blotting (E).

FIG. 5

Impact of flowrates and spiked samples: A) It was shown that 0.5 ml/hour gave the highest capture compared to 1 and 2 ml/hour. It was further shown that 200 HT29 cancer cells could be spiked into 1 ml blood and 4 ml blood, and captured with 51% and 47% efficiencies, respectively. B) After filtration, the filters were cultured for 21 days. Clearly, the captured cancer cells had expanded into clusters of cancer (bright areas in the center of the filter). The edge of the filter is depicted by a white circle. Scalebar, 2 mm

FIG. 6

During culture of the filtrated cells, the expansion of the cancer cells on the filter was followed by bright field microscopy: Panel A) after 14 days, clusters visible to the naked eye had emerged. Some clusters were more than 500 μm in diameter after 21 days of culture. The clusters could clearly be seen by the naked eye as seen by pictures acquired with a standard camera: Panel B) Here two filters are shown after 21 days of culture. The white arrows point out some of the cancer clusters.

FIG. 7.

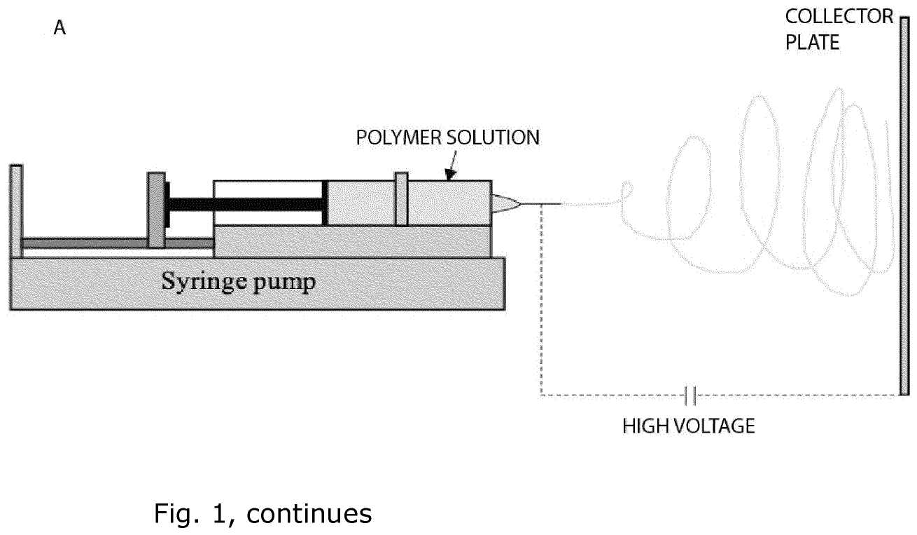

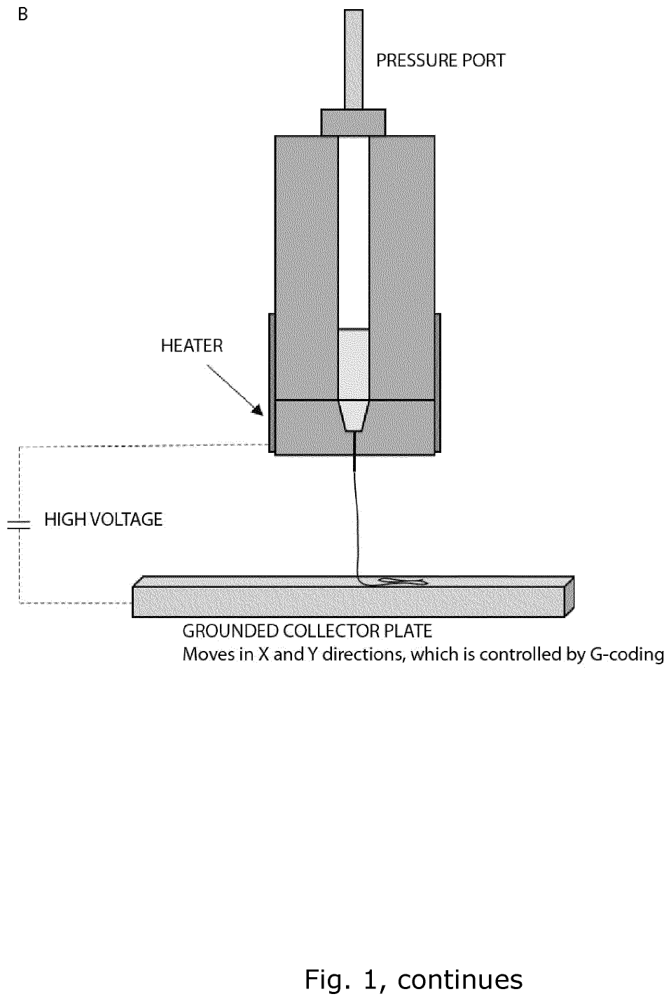

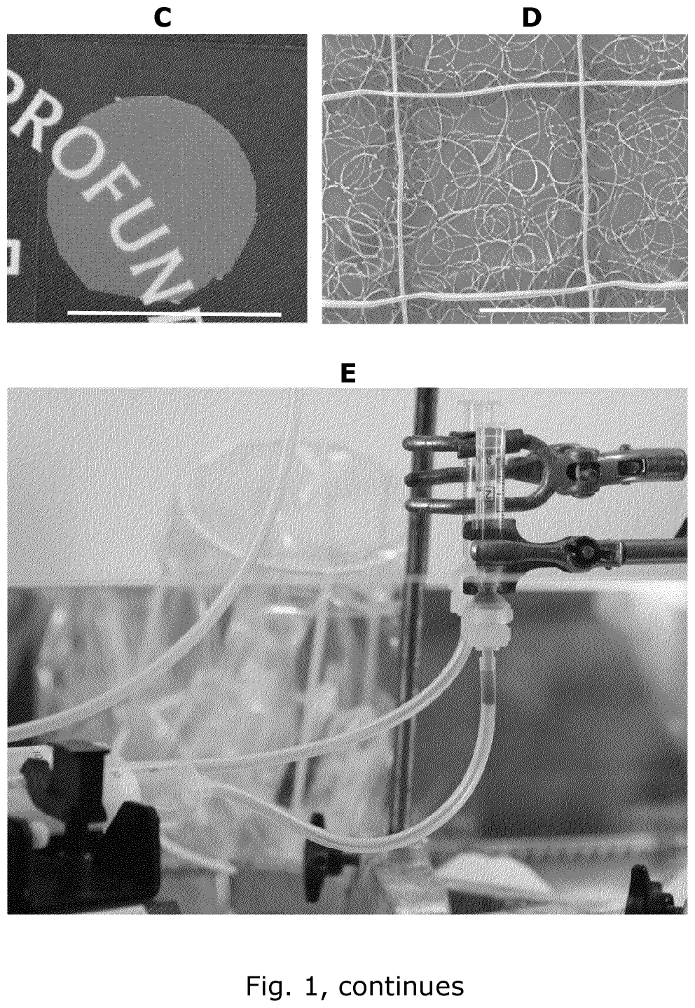

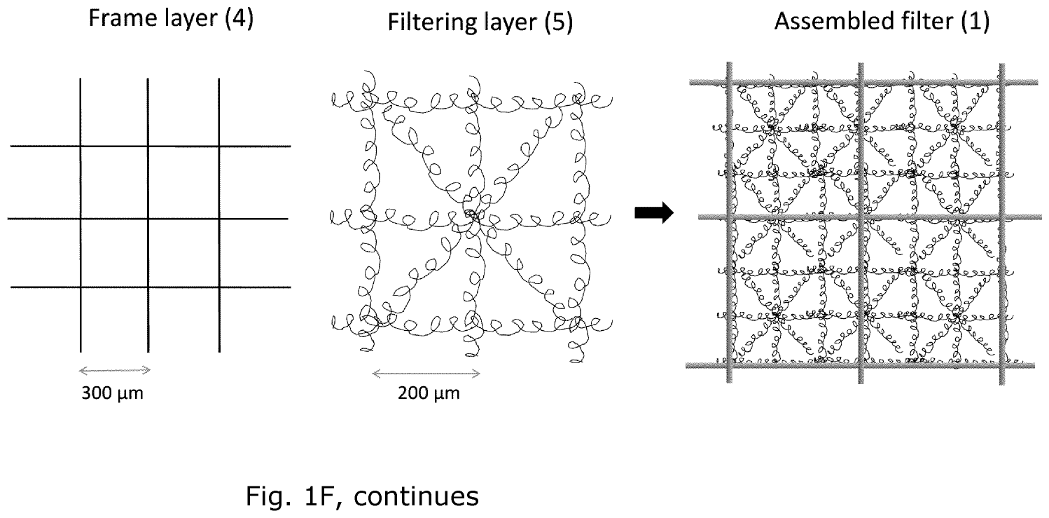

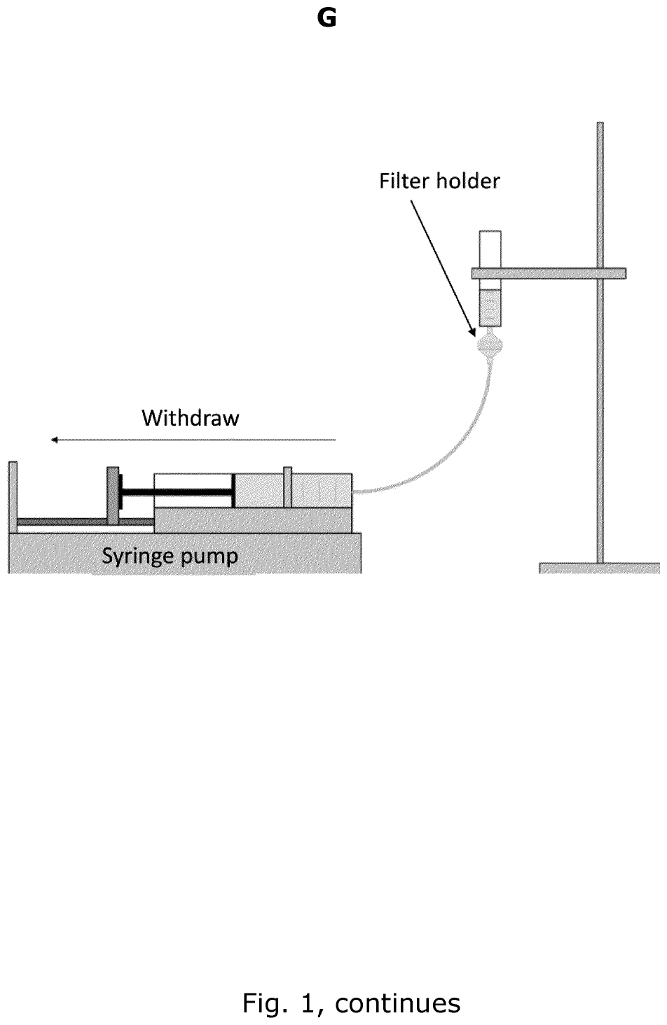

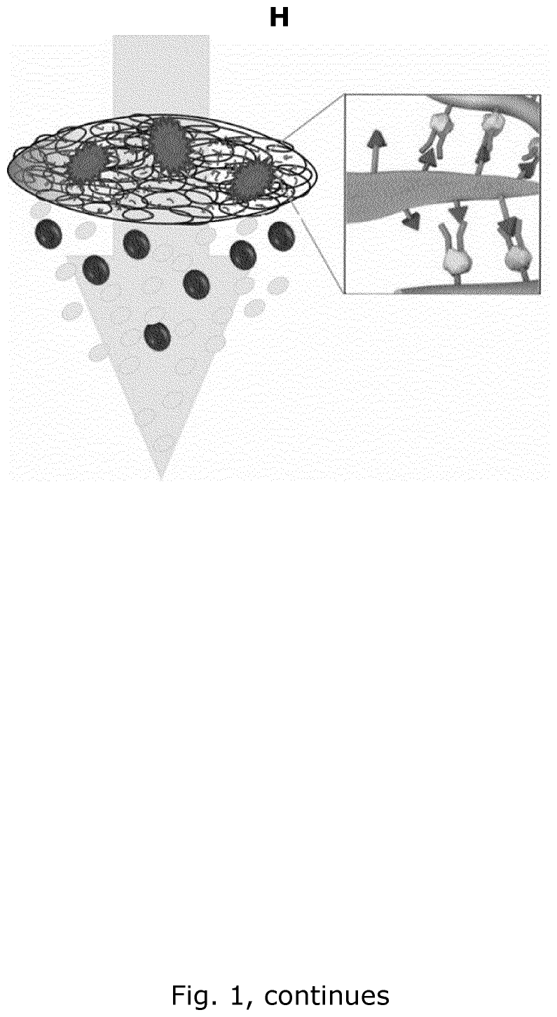

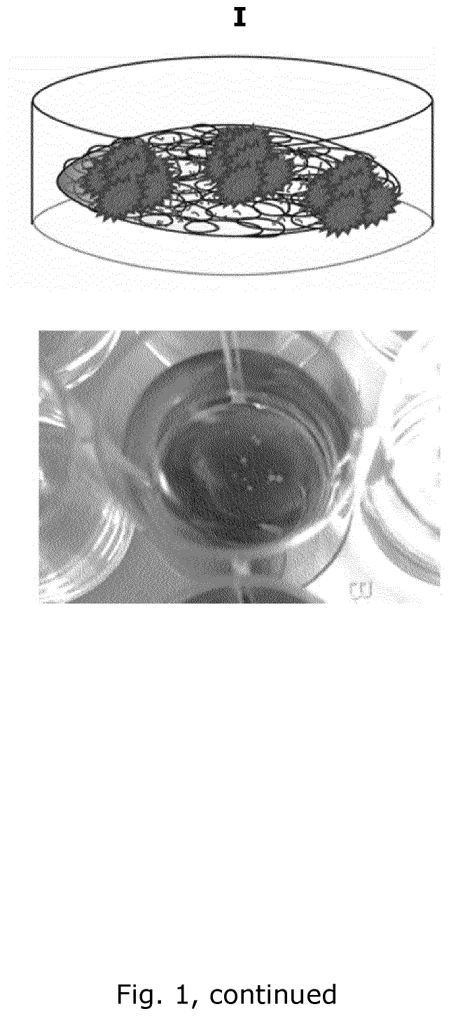

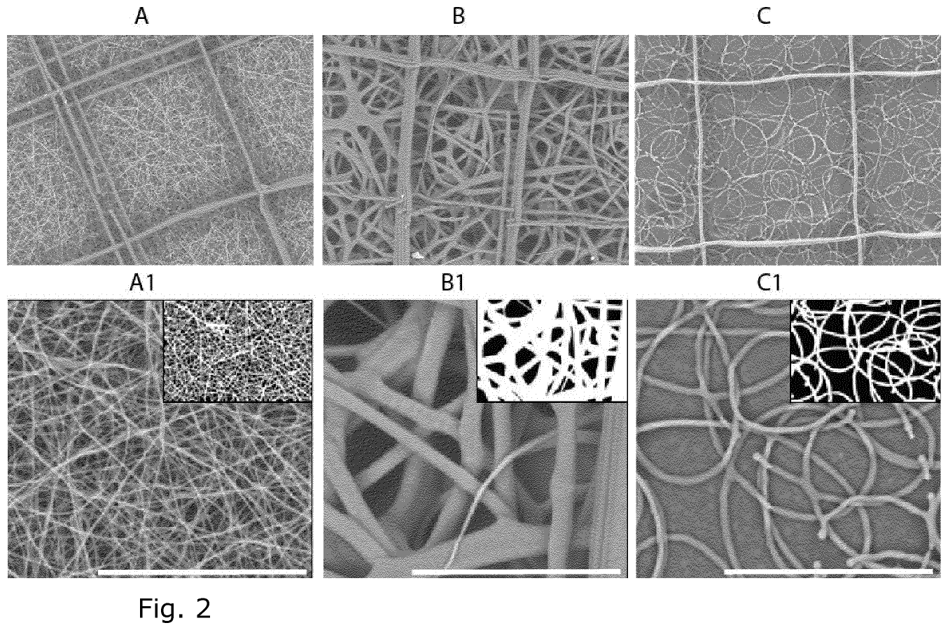

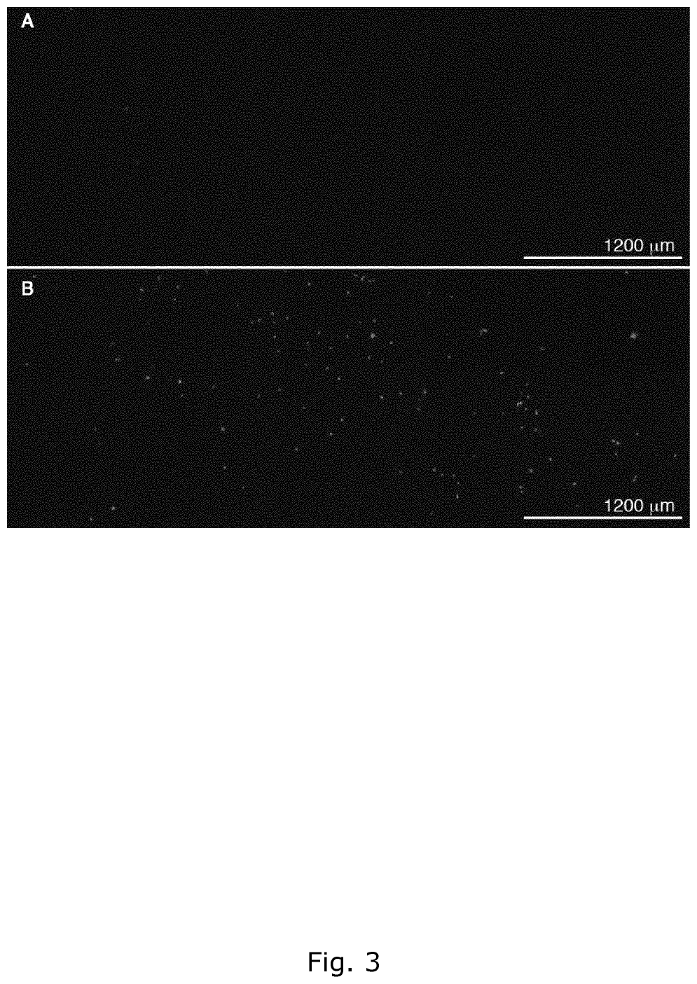

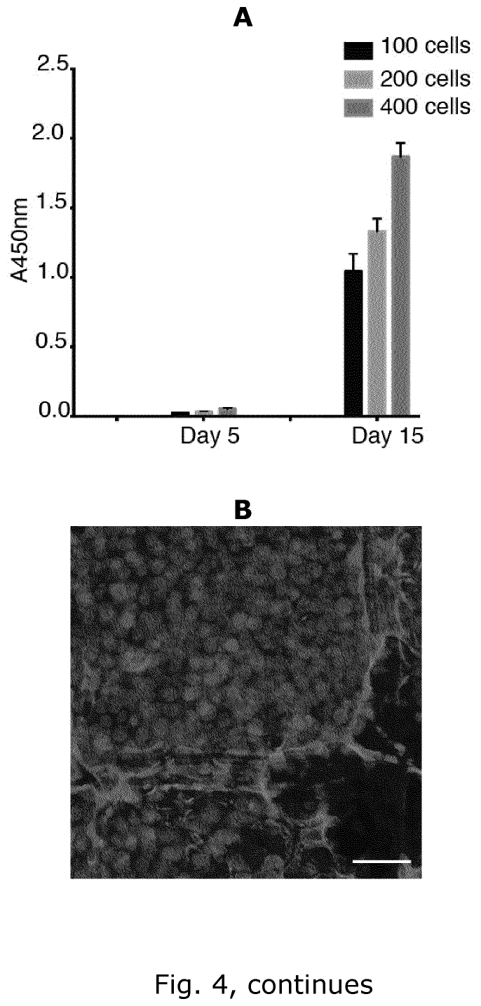

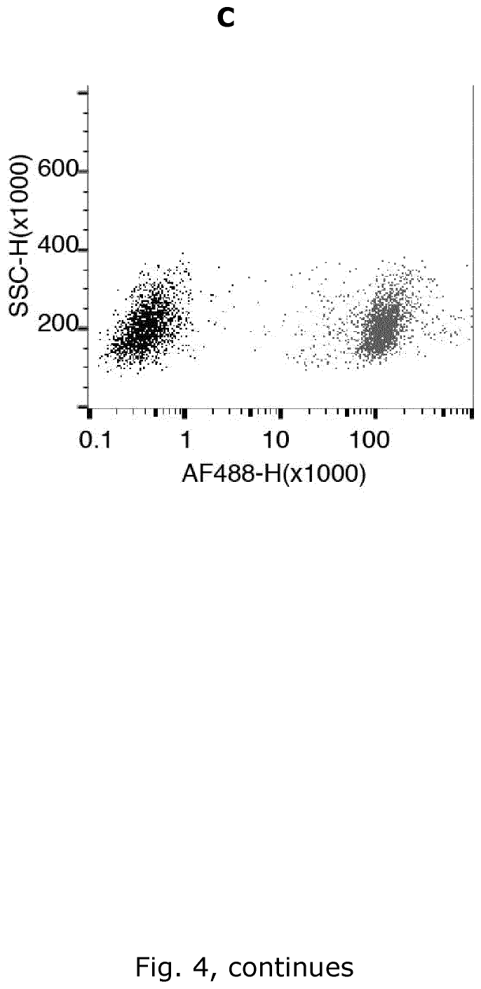

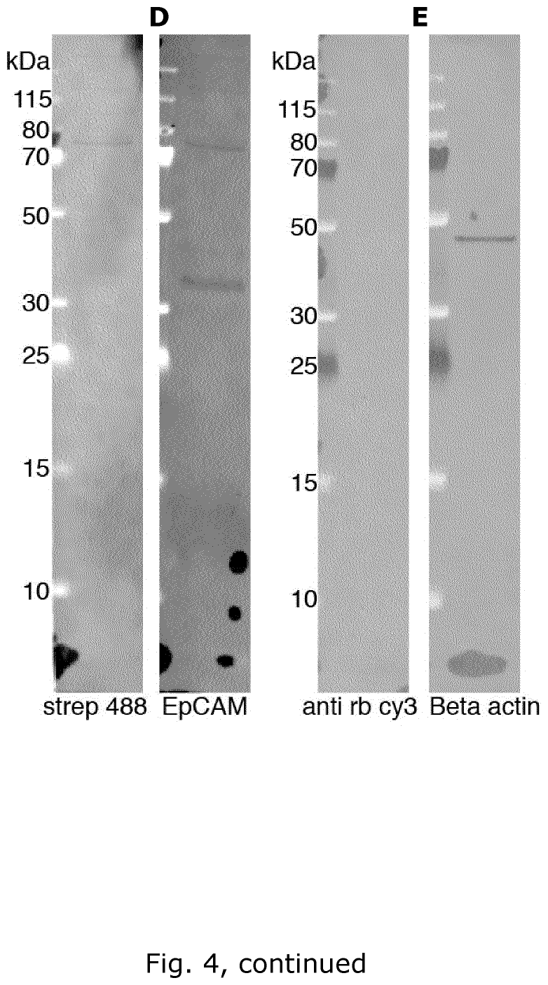

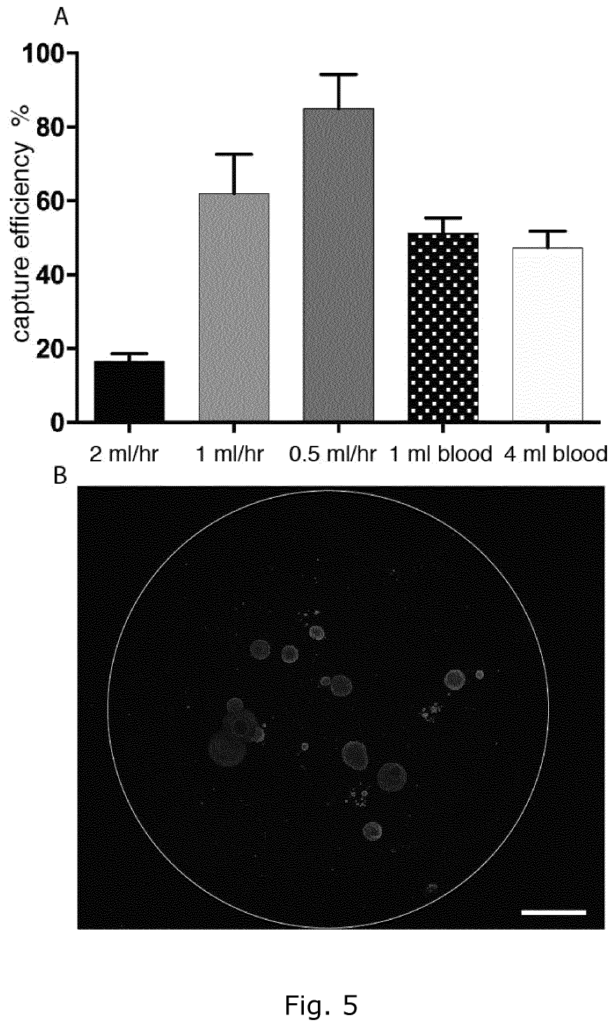

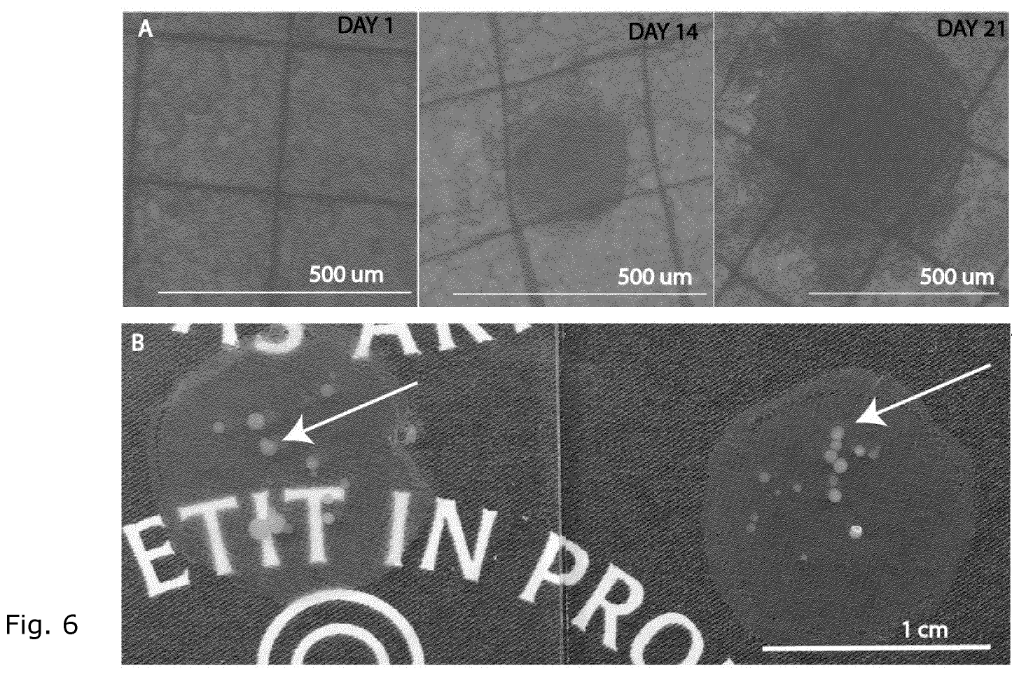

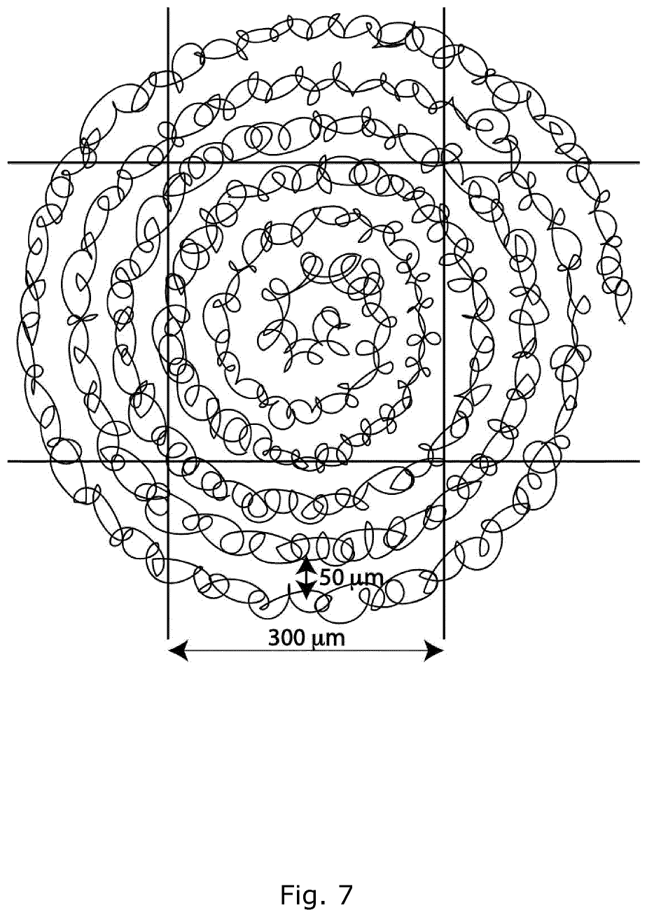

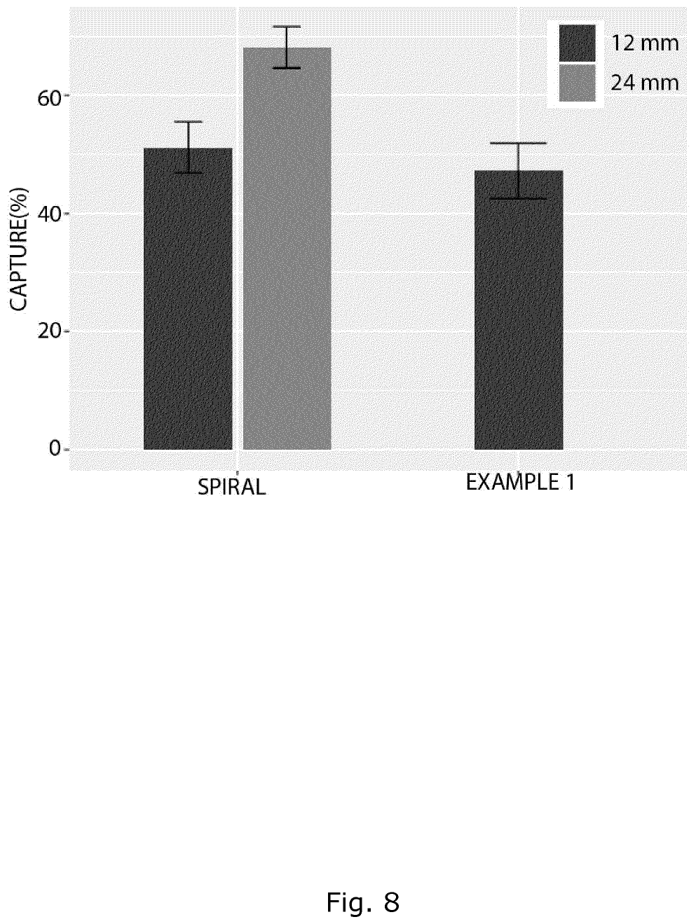

Schematics of a spiral filter design. Similar to the first design (FIG. 1, Example 1), the alternative spiral design is also composed of a 300×300 μm frame layer, and of a filtration layer. The fibers of the frame layer are written as straight lines (high printing speed, 2000 mm min−1), whereas the fibers of the filtration are coiled (low printing speed, 200 mm min−1). In the spiral design, the coiled filtration layer is written using a spiral pattern instead of horizontal, vertical and diagonal lines, and it has 50 μm spacing between the coiled lines of the spiral. This layer is approximately 10 μm of thickness.

FIG. 8.

The capture efficiency of the spiral design is comparable to the Example 1 showing 51.2% capture. Doubling the diameter from 12 and 24 mm of the filter increased the capture efficiency to 68.2% in the spiral design.

The present invention will now be described in more detail in the following.

### DETAILED DESCRIPTION OF THE INVENTION

### Filter

An improved filter suitable for filtering CTCs would be advantageous. In particular, a filter that can subsequently be used to culture the CTCs on, would be particular desired. Thus, a first aspect of the invention relates to a filter (1) comprising
a frame layer (4) of fibers (2); preferably comprising cell capturing moieties (3), preferably antibodies;
a filtering layer (5) of fibers (2), comprising cell capturing moieties (3), preferably antibodies; and

wherein the filtering layer (5) has a thickness in the range 1-100 μm.

As also outlined above, examples 1, 2 and 6 show and explain how such filter can be produced and how they are structured.

Filters with the properties according to the invention is difficult to produce. Thus, in an embodiment, the fibers (2) are fabricated by melt electrospinning writing (MEW), preferably PCL melt electrospinning writing (MEW) fibers. Melt electrospinning writing (MEW) is a processing technique to produce fibrous structures from polymer melts. In general, electrospinning can be performed using either polymer melts or polymer solutions. However, melt electrospinning is distinct in that the collection of the fiber can very focused. In the present context, the terms “Melt electrospinning”, “melt electrospinning writing” and “melt electrowritting (MEW)” are used interchangeably.

Preferably, both the filtering layer (5) and the frame layer (4) comprise cell capturing moieties (3). This is due to the filters are preferably coated after both layers have been produced. In principle, only the filtering layer could comprise the binding moieties, but from a practical point of view, the whole filter comprises the binding moieties.

The filtering layer (5), as outlined above, is very thin, therefore strength to the filter is applied by applying a stronger frame layer to the filter. Thus, in an embodiment, the filter has a thickness in the range 10-200 μm, such as 10-100 pm, or such as 10-50 μm. For example, the filter in examples 1 and 2, has a thickness of around 60 μm. Here the overall thickness comes from the frame layer (4) which may be comprised of several layers (such as 2-10) of fibers. The part of the filter where only the filtering layer (5) is present is of course thinner and the exact thickness at a specific point depends of the number of fibers crossing each other at that point.

Porosity of the filter is important to allow a fluid to pass through the filter in an efficient way, while still be able to capture the relevant cells. Thus, in another embodiment, the filtering layer (5) has a porosity in the range 30-80%, such as in the range 40-80%, preferably in the range 40-70% and more preferably 50-70%, such as 55-65%. In example 2, porosity has been tested for different filter types. In the present context, the term “porosity” refers to the summed area of the pores over the total area (summed area of both pores and fibers).

The size of the pores in the filter may also vary. Thus, in an embodiment, the filtering layer (5) has an average maximum pore axis, in the range 8-50 μm, preferably such as 10-20 μm, or more preferably such as 13-20 μm. In example 2, average maximum pore axis has been tested for different filter types. In the present context, the term “average maximum pore axis” refers to average of the maximum distances (edge to edge) of the pores including standard deviation.

In an embodiment, the filter is suitable for passing a fluid through the filter. This is in contrast to prior art (see background section), where material is positioned on a surface allowing fluid to pass over the material.

The diameter of the fibers have influence on the properties of the filter. Thus, in an embodiment, the filtering layer (5) has an average fiber diameter in the range 1-6 μm, preferably in the range 2.5-5 μm, more preferably in the range 2.5-4 μm. Again, in example 2, fiber diameter has been determined for different filter types. In the present context, the term “fiber diameter” refers to the average fiber diameter, the frame is not included.

The pore area of the filters may also vary. Thus, in a further embodiment, the filter (1) has an average pore area in the range 125-175 μm², preferably 135 -160 μm². Again, in example 2, fiber diameter has been determined for different filter types. In the present context, the term “average pore area” refers to the average area of the pores including standard deviation.

In an embodiment,
the frame layer (4) has a thickness in the range 10-100 μm, preferably 20-80 μm, even more preferably around 40-60 μm; and/or
the filtering layer (5) has a thickness in the range 2-50 μm, such as 2-40 μm, preferably 2-20 μm, even more preferably around 2-15 μm and/or
the filtering layer (5) has a thickness in the range 3-50 μm, such as 8-40 μm, preferably 5-20 μm, even more preferably around 5-15 μm.

In another embodiment, the filter has a diameter in the range 1-50 mm, such as 1-30 mm preferably 5-20 mm, more preferably in the range 8-15 mm, preferably in a circular shape. Such diameter will allow the filter to fit into standard filter holders.

In yet another embodiment, the filter has an area in the range 0.8-2000 mm² such as 0.8-700 mm² preferably 20-300 mm², more preferably in the range 50-200 mm², preferably in a circular shape. Such diameter will allow the filter to fit into standard filter holders. In example 7, circular filters with diameters of 12 mm (113 mm²) and 24 mm have been (452 mm²) showing that a high capture rate can be obtained with both filter types, but with a higher efficiency of the larger filters (FIG. 8).

As described above, the filter is very thin and does not have a 3D structure in the common understanding of the term. Thus, in an embodiment, the filter is a 2D filter.

To make a filter, a porous structure/form is introduced in the filtering layer (5). Thus, in an embodiment, the fibers (2) of filtering layer (5) have a coiled or nonlinear form. As also outlined in example 1, coiling can be introduced during production by controlling the printing speed.

In another embodiment, the fibers (2) of the filtering layer (5) have 10-100 coils per mm, such as in the range 10-70 coils per mm, preferably 10-50 coils, and more preferably 20-50 coils per mm, e.g. such as 20-40 coils per mm. The number of coils depends on the speed of spinning. Higher speed=fewer coils and vice versa.

The spacing of the fibers in the frame layer (4) has an influence on the overall strength of the fibers. Thus, in an embodiment, the fibers (2) of the frame layer (4) have a linear form, such as with a spacing around 100-500 um, such as 200-400 um.

The main foreseen use of the filters of the invention is capturing circulating cells, such as CTCs. Thus, in an embodiment, the filter (1) is for capturing cells, such as for capturing circulating cells, preferably circulating tumor cells (CTCs). “Circulating tumour cells” or “CTCs” are cells that have shed into the blood stream or lymphatics from a primary tumour and are carried around the body in the blood circulation. CTCs constitute seeds for the subsequent growth of additional tumours (metastases) in distant organs. The detection and analysis of CTCs can assist early patient prognoses and determine appropriate tailored treatments.

Thus, circulating tumour cells (CTCs) might be of great value for decision-making in the clinical management of cancer. However, the scarcity of CTCs in blood, particularly in that of colorectal cancer patients, is a serious bottleneck in the development of CTC-based precision medicine. In recent years, there has been an increasing appreciation of the establishment of CTC derived cell lines for further characterisation and drug testing (such as resistance to chemotherapeutics).

An advantage of electrospinning fibers is that a predetermined structure can be spun. Thus, in an embodiment, the fibers are positioned in a pre-determined pattern (reproducible pattern).

Different types of cell capturing moieties can be used on the filters of the invention. Thus, in an embodiment, the cell capturing moieties are antibodies or antigen binding fragments thereof, preferably antibodies such as monoclonal antibodies. In the example section, antibodies have been used (see example 3).

In a related embodiment, the cell capturing moieties are selected from the group consisting of binding moieties against biomarkers such as against EpCAM, surface vimentin, CD133, N-cadherin, EGFR, and cell surface biomarkers, such as cancer cell surface biomarkers.

It is noted that the cell capturing moieties can be bound to the fibers using different methods known to the skilled person. In example 3 streptavidin-polydopamine conjugation was primarily used but also EDC/NHS chemistry was tested. The first being the fastest method.

Different types of materials may be used for the fibers according to the invention. Thus, in an embodiment, the fibers consist of or comprise a material selected from the group consisting of Polycaprolactone (PCL), Polylactic acid, Poly(lactide-co-glycolide), Poly(methyl methacrylate), Polypropylene, Polyethylene, Poly(caprolactone-block-ethylene glycol) and Polyurethane, preferably the fiber comprises PCL, even more preferably the fiber consists of PCL. These polymers are examples of the most used polymers, and the skilled person may identify other fibers suitable for the filter according to the invention.

It would be advantageous if cells captured on the filter could be directly viewed (and mounted) in a light microscope. Thus, in an embodiment, the filter is transparent, such as for allowing for light microscopy analysis. FIG. 1C shows transparency of the filter of the invention.

In a further embodiment, the filter is suitable for in vitro growing cells thereon, such as for cell expansion. Example 4 and Example 5 show cell culturing directly on the filter of captured cells. This is believed to be an important feature of the filter of the invention.

### Method for Capturing Cells

The filters according to the present invention can be used for capturing cells, preferably CTCs. Thus, an aspect of the invention relates to a method for capturing cells, preferably circulating tumor cells (CTCs), the method comprising
a. providing a filter (1) according to the invention;
b. passing a sample suspected of comprising cells through the filter (1), thereby capturing the cells bound by the cell capturing moieties (3).

Example 5 and Example 6 outline how CTCs can be captured and cultured on a filter according to the invention.

The speed of the sample passing through the filter has an influence on the capturing rate of the filter. Thus, in an embodiment, the sample is passed through the filter (1) at a rate of 0.2-4 ml/hour, such as 0.2-2.5 ml/hour, preferably at a rate of 0.4-1.2 ml/hour, and more preferably at a rate of 0.4-0.8 ml/hour. In example 5, the capturing efficiency has been tested at different flow rates.

Different types of samples could be foreseen to be filtered by a filter according to the present invention. Thus, in an embodiment, the sample is a biological sample, preferably a blood sample, such as whole blood, blood serum or blood plasma, urine, or Cerebrospinal fluid (CSF). In example 5, CTC spiked blood samples were tested, confirming that the filters work as intended.

Thus, in another embodiment, the sample is suspected of comprising cells, such as circulating cells, preferably such as circulating tumor cells (CTCs).

In yet another embodiment, the CTCs are from solid tumor cancers, such as colon cancer CTCs, lung cancer CTCs, or breast cancer CTCs.

The sample volume passing through the filter during use may vary. Thus, in an embodiment, the sample volume is in the range 0.1-20 ml, such as 0.1-15 ml, such as 2-10 ml, such as 4-9 ml, preferably such as 6-9 ml, a more preferably such as 7-9 ml. In a related embodiment, the filter (1) is mounted in a filtering device, such as filter holder. The filter holder is required to stabilize the filter and to ensure that all sample fluid passes through the filter.

### Method for capturing and culturing cells

As outlined above, the filter according to the invention can be used for both capturing and culturing cells (such as CTCs). Thus, an aspect of the invention relates to a method for capturing and culturing cells, preferably CTCs, the method comprising
a) performing the capturing method according to the invention;
b) transferring the filter (suspected of comprising captured cells, preferably CTCs) to a culturing medium for the cells; and
c) culturing the cells on the filter, such as for at least 1 day in the medium.

Examples 4 and 5 show that cells can indeed be grown on the filter of the invention.

The cells can of course be cultured for different periods on the filters. Thus, in an embodiment, the step c) of culturing the cells are performed for a period of at least 5 days, such as at least 10 days, preferably at least 15 days, more preferably at least 20 days and/or in a period of 1-30 days, such as 10-30 days.

After culturing, the cultured cells can also be used for different further steps. Thus, in another embodiment, the method further comprises one or more steps selected from visual inspection of cells, coloring/labeling of cells, such as coloring/labeling of specific surface markers, such as cancer markers, testing cells for drug resistance, counting cells, flow cytometry, immunocytochemistry, western blotting, and storing/freezing of the cells.

### Process for Producing a Filter

The present invention also relates to a process for producing a filter according to the invention. Thus, yet an aspect of the invention relates to a process for producing a filter (1) according to the invention, the process comprising
melt electrospinning writing (MEW) a filtering layer (5) on a surface;
melt electrospinning writing (MEW) a frame layer (4) on the filtering layer (5);
linking (conjugating to) at least the filtering layer (5) (preferably both layers) with cell capturing moieties (3); and
providing a filter (1) according to the invention.

Although in the examples, the filtering layer was produced before the frame layer, it is to be understood that the steps could also be performed in an opposite order. Thus, in an alternative aspect the frame layer (4) is melt electrowritten before the filtering layer (5) is melt electrospun written on the frame layer.

In an embodiment, the frame layer (4) and filter layer (5) are melt electrowritten in a predetermined pattern. In a similar embodiment, the frame layer (4) and filter layer (5) are melt electrowritten in a reproducible pattern.

As also outlined above and in the example section, the speed of electrowriting and the needle diameter may influence how the final structure of the filter appears. Thus, in an embodiment,
the filtering layer (5) is melt electrowritten at a speed in the range 100-700 mm min⁻¹, such as in the range 100-500 mm min⁻¹, preferably in the range 300-500 mm min⁻¹, and even more preferably 350-450 mm min⁻¹; and/or
a needle with a diameter in the range 0.1-0.5 mm is used, preferably 0.2-0.4 mm in diameter and more preferably in the range 0.25-0.35 mm in diameter, such as around 0.3 mm in diameter.

In a related embodiment, the filtering layer (5) is melt electrowritten in straight lines in perpendicular directions and diagonal directions, at a distance in the range 100-300 μm, preferably 150-250 μm, even more preferably in the range 175-225 μm, such as around 200 μm (see also FIG. 1F, middle and right panel).

In yet an embodiment, the filtering layer (5) is melt electrowritten in a spiral shape with coiled fibers (Example 7, FIG. 7). In yet an embodiment, there is a distance between the coiled lines of the spiral in the range 10-100 μm, such as 20-80 μm, such as 30-70 μm, or such as 40-60 μm, or such as around 50 μm. Again, this spiral design is combined with a frame layer as also shown in FIG. 7. As also explained in example 7, the spiral design provides increased capture efficiency and decreased production time compared to the design of example 1. It is of course to be understood that both designs works for the purpose of capturing, culturing and imaging cells on the filter.

In another embodiment,
the frame layer (4) is melt electrowritten at a speed in the range 1000-3000 mm min⁻¹, such as in the range 1500-2500 mm min⁻¹, preferably in the range 1750-2250 mm min⁻¹, and even more preferably 1800-2200 mm min⁻¹; and/or
a needle with a diameter in the range 0.3-0.7 mm is used, preferably 0.4-0.6 mm in diameter and more preferably in the range 0.51-0.59 mm in diameter, such as around 0.55 mm in diameter.

In a related embodiment, the frame layer (4) is melt electrowritten in straight lines in perpendicular directions, at a distance in the range 100-500 μm, preferably 200-400 μm, even more preferably in the range 250-350 μm, such as around 300 μm (see also FIG. 1F, left and right panel).

Since the filters may be used for culturing the captured cells, it is an advantage if the filter is sterile. Thus, in an embodiment the process includes a sterilization steps, such as before conjugation/coating of the fibers. Sterilization could e.g. be by ethanol and/or UV light.

### Filter Obtained/Obtainable

The filters according to the present invention are produced by a specific method according to the invention. Thus, in a further aspect the invention relates to a filter (1) obtained/obtainable by the (manufacturing) process according to the invention.

### Devices

The filter according to the invention can form part of other devices. Thus, an aspect of the invention relates to a filtering device, such as a filter holder comprising a filter (1) according to the invention.

Another aspect relates to a cell culturing device comprising a filter (1) according to the invention. In an embodiment, the filter (in the cell culturing device) comprises viable cells, preferably derived from CTCs.

A further aspect relates to a microscope comprising a filter (1) according to the invention, positioned for microscopy analysis. In an embodiment, the filter (in the microscope) comprises viable cells, preferably derived from CTCs.

### Kit

The filter according to the invention can also be part of a kit of parts. Thus in an additional aspect the invention relates to a kit of parts comprising
a filter (1) according to the invention;
a filter holder for the filter;
optionally, buffers for anticoagulation;
optionally, one or more syringes; and
optionally, instructions for use.

In an embodiment, the instructions for use relates to the use of capturing and/or culturing cells according the corresponding aspects of the invention.

### Uses

The filters according to the invention may have different specific uses. Thus, yet another aspect of the invention relates to the use of a filter (1) according to the invention, for filtering a sample, such as a sample selected from a biological sample, preferably a blood sample, such as whole blood, blood serum or blood plasma, urine, and cerebrospinal fluid (CSF).

A further aspect of the invention relates to the use of a filter according to the invention, for (in vitro) capturing and optionally culturing cells, preferably CTCs.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Production of the Filters

### Aim of Study

To produce cell capture filters by electrospinning technologies.

### Method

Electrospinning is a technique where viscous polymers can be “spun” from a needle as ultrathin fibres and collected on a solid surface or in a solution. In “solution electrospinning” a polymer is dissolved in a solvent and pushed through a small needle. Under normal circumstances, the polymer will eventually start to come out of the needle as drops, however in electrospinning a high voltage potential is applied between the needle and the collector. This forces the polymer to spin out of the needle as a jet towards the collecting surface. In melt electrowriting (MEW), the same principle is applied however instead of dissolving the polymer, the polymer is melted inside the needle by raising the temperature. The melted polymer is then pushed out of the needle by applying pressure from the top. Polycaprolactone (PCL) for the filters of the invention has been used. In total three different filters have been tested, two produced by solution electrospinning (FIG. 1A) and one by MEW (FIG. 1B). The solutions used for solution electrospinning were:
PCL dissolved in chloroform (CF);
PCL in a combination of dichloromethane (DCM); and N,N-dimethylformamide (DMF).

These are referred to as CF and DCM in table 1 (See example 2).

The MEW technique is preferred for the present invention, because it allows for more controlled “writing” of the fibres compared to solution electrospinning techniques and MEW also adds other appreciated features to the filter (See example 2). The produced MEW filter can be seen in FIG. 1C. In FIG. 1D, the fibres can be seen at high magnification: the frame layer (4) is made from the thick linear fibres whereas the filtering layer (5) is made from the coiled fibres. The filtration setup, where cancer cells in blood are captured, is shown in FIG. 1E. Further, in FIG. 1F a schematic overview of the filter according to the invention is presented. The MEW filter is assembled from a frame layer (4) made from the thick linear fibres for stabilizing the filter (FIG. 1F, left) and a filtration layer (5) made from coiled fibres for size-dependent filtration (FIG. 1F, middle). Together the two layer constitutes a filter (1) according to the present invention (FIG. 1F, right).

In FIG. 1H flow through a filter is illustrated, where CTCs are captured by the filter, whereas normal blood cells passes through the filter. The insert in FIG. 1H illustrates how capturing moieties on the filter binds to epitopes on the CTC surface. After filtering, the filter can be moved directly to a culturing medium as illustrated in FIG. 1I (top). After culturing for a period of time (such as up to three weeks), cultured cancer cells are visible (FIG. 1, bottom).

Thus, a preferred use of the filter according to the invention is filtering of circulating tumor cells (FIG. 1H). The whole filter is further modified with antibodies (cell capturing moieties (3)) for affinity-based filtration (FIG. 1H). The modified filter is then mounted onto a filter holder, where the holder is attached to a syringe onto which the blood sample is loaded. The syringe and the filter holder is connected to a withdrawing syringe pump for flow-rate controlled filtration (as seen in FIG. 1G). Right after filtration, the cell-caught filter can be taken out from the filter holder and directly incubated in standard cell culture condition (as seen in FIG. 1I). It is seen that the few caught cells can easily expand over a couple of weeks to visible cancer bodies/subcolonies (as seen in FIG. 1I, bottom).

For detailed description of filter production, please see Example 6.

### Conclusion

The MEW filter fits in a simple filtering setup, it is transparent, and the method is reproducible.

### Example 2—Characterisation and Assessment of Filtration Capacity

### Aim of study

To test the scaffold's suitability for filtering samples holding white blood cells, and to characterise the filters.

### Materials and Methods

See Examples 1 and 6, if not otherwise described.

### Filtration of White Blood Cells and Characterisation

Four million white cells were filtered through each filter type (CF, DCM and MEW) at 4 ml/hour, the cells were fixed and then stained with phalloidin af488. For determination of filter characteristics, SEM images were analyzed by using the DiameterJ plugin in ImageJ software. SEM images of the different filters were acquired with the same magnification and used for analysis in image J.

### Results

It has previously been demonstrated that PCL dissolved in chloroform (CF) can be transformed into highly biocompatible fibers by electrospinning. It has also been shown that PCL in a mixture of dichloromethane (DCM) and N,N-dimethylformamide (DMF) can be extruded to unique biocompatible fibers by electrospinning. Consequently, it was attempted to make improved filters using similar approaches. In parallel, the MEW technique was explored to make a PCL filter.

Initial filtrations were performed on white blood cells using the different filters. There are billions of blood cells and only a few CTCs in the blood of colorectal cancer patients, and it might be beneficial to decrease the amount background blood cells to ease downstream culture and analysis. In the initial filtrations, the MEW filter captured fewer white blood cells compared to the CF and the DCM filters. Furthermore, challenges with reproducibility and reduced transparency of the CF and DCM filters were encountered when performing downstream analysis by microscopy.

Characteristics of the three filter types were determined by using the DiameterJ plugin for imageJ (table 1 and FIG. 2).

TABLE 1

| Metric | DCM | CF | MEW |
|---|---|---|---|
| Pore axis (μm) | 4.1 ± 2.0 | 15.4 ± 8.4 | 16.3 ± 9.9 |
| Pore area (μm²) | 7.4 ± 6.3 | 112.8 ± 118.9 | 141.5 ± 146.8 |
| Porosity (%) | 50 | 25 | 60 |
| Fiber diameter (μm) | 0.9 ± 0.2 | 7.7 ± 2.5 | 3.3 ± 0.45 |

Pore axis: average maximum distances (edge to edge) of the pores, including standard deviation
Pore area: the average area of the pores including standard deviation.
Porosity: summed area of the pores over the total area.
Fibre diameter: average fibre thickness, the frame layer fibres are not included.

### Conclusion

Although the DCM and CF filter versions, might have potential for culture of CTCs, they failed in at least three perspectives. Firstly, it was difficult to reproduce the filters as they often had different thicknesses. Secondly, they caught more of the white blood cells in the preliminary test filtrations than did the MEW filter. Thirdly, the DCM and CF filter versions had problems with transparency.

In sum, the MEW filter showed superior qualities in respect of capturing the least of the white blood cells attached to the filters thereby minimizing background. Furthermore, the MEW filter is reproducible and transparent.

### Example 3—Bioconjugation of the MEW Filter

### Aim of Study

To test impact of the anti EpCAM antibody on the capture of cancer cells.

### Materials and Methods

See example 6, if not otherwise described.

### Results

The MEW technique was used to make scaffolds that enable capture and in situ culture. This filter is transparent and it can be used with standard filter holders. Such holders can easily be connected to syringes and the flow through the filter can be controlled by using a syringe pump (FIG. 1E).

For isolating CTCs of epithelial origin, the EpCAM surface molecule has become a golden standard. Accordingly, to test the compatibility of the MEW filter with bioconjugation, a polydopamine based conjugation of streptavidin was performed and then a conjugation of biotinylated anti EpCAM antibody to the PCL fibers. Also, we conjugated the antibody to the filter via EDC/NHS chemistry but as this takes a longer time and since there's no difference in capture efficiency, we primarily used the polydopamine based conjugation.

We tested the whole setup and compared the capture of 2000 HT29 colon cancer cells on a non-conjugated (no antibody) filter with a conjugated filter (with anti EpCAM antibody). It was shown that the presence of the antibody is important for the capture of the cancer cells. (FIG. 3)

### Conclusion

The presence of capture moieties (3) such as antibodies is important for capturing cancer cells. The capturing moieties can be conjugated to the fibers by different methods.

### Example 4—Testing Whether MEW Filter is Suited for Cell Culture and for Proteomic Downstream Analysis

### Aim of Study

To verify that a low number of cancer cells can indeed be cultured on the MEW filters and to assess if these cultured cells subsequently can be analysed by flow cytometry and western blotting.

### Materials and Methods

See example 6, if not otherwise described.

### Results

As CTCs are rare in blood, a filter/scaffold that enables culture of these rare events are desirable. It was therefore assessed whether a few cells could be expanded on the anti EpCAM conjugated MEW filter by seeding 100, 200, and 400 HT29 cells per scaffold. The cells were expanded on the filters/scaffolds within two weeks, as shown by cck-8 assay measurements and immunochemistry (FIG. 4A and 4B). It was also tested whether cells from the filters could be analyzed by common proteomic methods such as flow cytometry (FIG. 4C) and western blotting (FIG. 4D and 4E).

The expanded cells were detached from the filter by trypsin treatment, and flow cytometry was successfully performed on the cells. The cells were gated according to their forward versus side scatter. The signal of a non-stained sample from one scaffold was then compared to the signal of anti EpCAM stained cells detached from another scaffold, and a clear shift was observed (FIG. 4C). Additionally, cell lysates were obtained from cultured filters/scaffolds on which EpCAM (FIG. 4D) and beta actin (FIG. 4E) could be detected by western blotting.

### Conclusion

Cancer cells can grow effectively on the filters of the invention and these can be analysed by standard proteomic approaches.

### Example 5—Testing the Impact of Flow Rate, Filtration of Clinical Relevant Samples and Culture of Filtrated Cancer Cells

### Aim of Study

To test different flow rates, to perform filtration of clinical relevant samples and to culture filtrated cancer cells.

### Materials and Methods

See example 6, if not otherwise described.

### Results

It is shown that the flow rate by which the sample is filtrated has a high impact on the capture efficiency. 2 ml/hr, 1 ml/hr and 0.5 ml/hr were tested, where the capture efficiencies were 16.5%, 62%, and 85% respectively (FIG. 5A). Next, the capacity of the filter was tested on clinical relevant spiked-in-samples, where 200 HT29 colon cancer cells were spiked into 1 ml and 4 ml whole blood, respectively. Triplicate measurements were conducted. For the “1 ml blood” spike-in-sample, 51% of the cells were captured, while 47% percent was captured in the “4 ml blood” spike-in-sample, both performed at 0.5 ml/hour (FIG. 5A). It was then assessed whether 200 colon cancer cells, spiked into 4 ml whole blood, could be filtrated, captured, and cultured on site (FIG. 5B and 5C). The expansion of single cancer cells into cancer clusters was followed by bright field microscopy, where it was observed that clusters visible to the naked eye had emerged after 14 days (FIG. 6A). The experiment was ended after 21 days, and pictures of the filters were taken with a standard camera, clearly demonstrating that the cancer clusters were visible to the naked eye (FIG. 6B).

### Conclusion

It has been demonstrated that the MEW filter can be used as a capture and culture device, here illustrated for culturing rare cancer cells of the blood.

### Example 6—Detailed Materials and Methods

### Electrospinning

Two PCL based filters were made by using solution electrospinning. Two PCL solutions were made: A 12% weight/volume PCL solution was made by dissolving PCL (M_{w}: 80 kDa) in dichloromethane (DCM) and N,N-dimethylformamide (DMF) (3 parts DCM and 2 parts 2 DMF), and a 17.5% weight/volume PCL solution was made in chloroform (CF). Dry electrospinning was performed horizontally onto foil at a distance of 12 cm from the blunt end of a 16G needle to a collector. The voltage was 15 kV. A flowrate of 1 ml/hour and 3 ml/hour was used for DCM/DMF and CF, respectively. The DCM/DMF solution was electrospun for 15 minutes while CF was spun until desired density was reached. A frame was made on top of both filters using Melt electrospinning machine with PCL (M_{w}: 45 kDa), before filters were punched out as 12 mm diameter filters.

### Filter Characterisation

The DiameterJ Segment was chosen under Plugins, for a segmentation analysis. Traditional Segmentation Algorithm was chosen and performed. After the segmentation analysis, three folders containing the images were created: Best Segmentation, Montage Images, and Segmented Images. The best segmentation of each image was determined using either the montages or the segmented images alone and the original image. The best segmented match was opened in ImageJ along with the original image, and the images were overlaid with an opacity of 75%. The segmented images were then manually adjusted to distinguish background (black) and fibers (white) from each other (FIG. 2: A1, B1, and C1), to match the original image perfectly. The overlay was removed before the image was saved in the folder called Best Segmentation. The final analysis was carried out using DiameterJ 1-018 in the DiameterJ plugin.

### Melt electrospinning Writing and Preparation of Filter

Melt-electrospinning writing (MEW, CAT000111, Spraybase) was performed to print the filters. Polycaprolactone (PCL, M_{w}=45 kDa, Sigma-aldrich) was loaded in a grounded stainless steel syringe, and heated to 80° C. Then a gas pump was used to supply air pressure of 0.5 bar for extruding the melted PCL polymer out from the syringe connected with a blunt-end stainless steel needle. An automated plate collector was placed under the needle, and connected to a high voltage of 3.5 kV. Movement of the collector was precisely controlled by UCCNC software. The distance between the needle and collector was 4 mm. Coiled fibers were first printed as the filtering layer (5) (FIG. 1D and 1F) at 400 mm min⁻¹ using a 0.3 mm in diameter needle. The relatively low speed facilitated coiling of the fibers. The coiled fibers were printed in two perpendicular directions and in their two diagonal directions with the same spacing distance. This formed a porous monolayer membrane of coiled fibers. Afterwards, a frame layer (4) (FIG. 1D and 1F) composed of bigger straight fibers for stabilizing the underneath membrane was printed on top of the filtering layer at a 2000 mm min⁻¹. The top layer needle had a diameter of 0.55 mm, and the spacing was 300 μm. The top frame layer was prepared by printing straight fibers in two perpendicular directions.

After electrospinning, filters were transferred to a 70% EtOH bath from where they were mounted to glass slides and with a scalpel cut to fit the filter holders (Millipore, cat no. SX0001300).

In sum, the MEW filter holds 30 fiber coils per mm, which were written in the x and y directions and in two diagonal directions with 200 μm spacing. Additionally, a frame layer for stabilization was printed on top of the three types of filtering layers of the three different filters in the x and y directions with 300 μm spacing. Characteristics of the three filter types were determined by using the DiameterJ plugin for imageJ (table 1 and FIG. 2A, A1, B, B1, and C, C1).

### Bioconjugation of a Biotinylated Anti Human EpCAM Antibody to the Filters

If the filters were used for culture, they were placed in a drop of 70% ethanol prior to bioconjugation and the ethanol was evaporated in a sterile laminar flow bench. A UV source (around 260 nm) of a sterile bench was then used for additional sterilisation for more than one hour. All buffers were autoclaved or sterile filtered (0.2 μm).

All conjugation steps were performed at room temperature. The anti EpCAM antibody was conjugated to a filter via polydopamine coating. In short, the filter was kept on glass slides (VWR, cat no 631-1551) within an area encircled using a liquid blocker super PAP pen (Fisher scientific, cat no. NC 9827128). Dopamin hydrochloride (Sigma, cat no. H8502) was added (2 mg/ml in 500 μL 10 mM Tris-HCL, pH 9) to the filter for minimum 30 minutes, making sure that the filter was soaked with the dopamine solution hereby creating a polydopamine surface. Next, the filter was washed four times in 400 μL sodium phosphate buffer (50 mM, pH 7.8). Streptavidin(15 μg/mL) was then conjugated to the polydopamine layer for minimum 45 minutes in sodium phosphate buffer. The filter was washed four times in phosphate buffered saline(PBS, Sigma, cat no. P4417), before transferring the filter to a fresh glass slide. Here, the filter was incubated with 5 μg/mL of biotinylated anti EpCAM antibody (RnD Systems, BAF960) in 1% bovine serum albumin in PBS for more than an one hour. Finally, the filter was washed in PBS four times.

Alternatively, the conjugation can be performed by classical EDC/NHS chemistry on pretreated filters (50 mM NaOH, 5 min, rinsing in PBS), incubating with EDC(2 mg/mL) and NHS(8 mg/ml) in MES buffer pH 6. Then using streptavidin at 125 ug/mL and anti EpCAM antibody at 10 ug/mL.

### Immunocytochemistry

In general, scaffolds and cells were stained on VWR glass slides (cat no. 631-1551) or on cover slides (VWR, cat no. 631-0138). The cells or scaffolds were placed in an encircled area by using the aforementioned PAP pen to create a hydrophobic barrier. Next, samples were washed in DPBS or PBS, and fixed in 4% formaldehyde(AppliChem, cat no. A3813), followed by four times rinsing in PBS. The cells were then permeabilised and blocked in BD Perm/Wash buffer (BD Biosciences, cat. 554723) for 10 minutes, before adding primary antibodies or phalloidin alexa fluor 488 (ThermoFisher Scientific, cat 12379) in Perm/Wash buffer. The primary antibodies mouse monoclonal anti pan cytokeratin (ab86734, abcam) and rabbit recombinant anti CD 45 (ab40763, abcam) were used in 1:200 and 1:100 ratios, respectively, and incubated 2 hrs at room temperature or overnight at 4° C. Phalloidin alexa fluor 488 was used in 1:100 ratio for one hour staining and 1:400 for overnight staining. Next, washing was performed three times, three minutes each, in 400 uL Perm/wash buffer. Finally, the secondary antibodies anti mouse and anti-rabbit alexa fluor 488/594 (Abcam, cat no. ab150105, ab150076) was used 1:400 in Perm/Wash buffer for 1 hour at room temperature. The washing was repeated. Hoecsht (Life Technologies, cat no. H3570) was then used 1:10,000 in PBS for 5 minutes, the sample was rinsed, gently desiccated, and mounted with aqueous mounting media (Sigma, cat no. F4680). Staining was analyzed in EVOS FL Auto Imaging system or Zeiss confocal laser scanning microscopy (LSM 700 and 800).

### Cell Seeding on Scaffolds and Cell Counting Kit-8 Measurements

Prior to cell culture, all filter scaffolds were conjugated to the anti EpCAM antibody under sterile conditions. HT29 cells were seeded to the scaffolds in 20 uL media in a TC 96-well culture plate (SARSTEDT, cat no. 83.3925.500) in different densities (100, 200, and 400 cells per scaffold, four replicate for each seeding density). After one hour, additional 180 uL media were added to the scaffolds. The next day (day 1), each scaffold was transferred to new wells, fresh media was added, and the scaffolds were left there until day 5 where the cell viability was assessed by using the Cell Counting Kit-8 as outlined by the manufacturer (Dojindo Molecular Technologies). The Cell Counting Kit-8 was applied again on day 15, each time the measurements were performed in new wells to rule out any signal from cells not growing directly on the scaffolds. After finishing the cell counting on day 15, the scaffolds were rinsed in PBS, and the presence of cells was confirmed by immunocytochemistry using the anti-pan cytokeratin antibody and Hoechst stain as outlined above.

### Pre-Staining HT29 With Cell Tracker Dye

Approximately five million HT29 cells were washed twice in DPBS (sigma, cat). Cells were centrifuged at 300 g for 3 minutes each time. The cells were re-suspended in DMEM without fetal bovine serum but holding 1 μM CellTracker Red CMPTX dye (ThermoFisher, cat no. C34552). After 30 minutes in the CellTracker solution at 37° C., the cells were washed twice in complete growth media and kept in media until further use.

### Processing Blood Samples

Eight milliliters of blood from healthy volunteers were drawn into EDTA coated tubes (BD). The blood was processed within 6 hours. Lysis of red blood cells was performed by mixing 4 ml blood with 40 ml lysis buffer(155mM NH₄Cl, 12mM NaHCOO₃, 0.1 mM EDTA), it was incubated for 10 minutes at room temperature followed by centrifugation (5 minutes, 1000 g). If necessary, the cells were resuspended, and the centrifugation was repeated. Without disturbing the pellet, the supernatant was gently aspirated and PBS was added to total a volume of 1.5 ml.

### Spike-In Experiments

For whole-blood samples with non-lysed red blood cells, 200 pre-stained HT29 were added to 1 ml whole blood and filtrated. For samples where red blood cells were lysed, 200 pre-stained HT29 cells were added to 4 ml whole blood, and the red blood cells were lysed as described above. The flow rate was 0.5 ml/hour

### The Filtrations

A filter was transferred to a sterile (if needed for culture) water bath and the floating filter could be mounted from underneath directly to the bottom part of a filter holder. The top part of the filter holder was gently attached to the bottom part, and through a tubing connected to a 20 ml syringe placed in a syringe pump (World Precision Instruments, cat no. 941-371-10003). Next, the filter holder was carefully filled with 10% FBS in PBS, taking care that air was not trapped above the filter. The filter was left like this for 10 minutes before commencing the filtration. A 3 ml syringe was then connected to the top of the filter holder, and the sample was gently loaded to this syringe. The syringe pump was set to withdraw mode. Just before the entire sample had run through the filter, additional 300 μL PBS were added for washing. The immunocytochemistry was performed directly on the filter holder, and the cells could be visualized while still being placed on the filter holder by gently placing the bottom part of the filter holder directly on top of a cover slide.

### Cell Lysing and Western Blotting

Four hundred HT29 cells per scaffold were seeded. Each scaffold was conjugated the anti EpCAM antibody. The cells were cultured for 28 days, where after the cells, still on the scaffolds, were washed in DPBS 2-3 times and could be kept at −20° C. until further use. To lyse the cells on the scaffolds, 100 μL per scaffold of M-PER solution (Thermo Scientific, cat no. 78503) were used. After 10 minutes incubation at room temperature, the lysates were centrifuged 15 minutes at 14,000 g. The supernatant was harvested. To prepare samples for SDS PAGE, 80 μL of each sample were mixed carefully with 20 μL XT sample buffer(BIORAD, cat no. 161-0791) and 6 μL XT reducing agent (BIORAD, cat no. 161-0792). The samples were left at 90° C. for 5 minutes. Next, 20 μL of each sample were loaded to a 12% XT Bis-tris gel (BIORAD, cat no. 3450118) and run at 200 V for 50 minutes. Proteins were blotted to a nitrocellulose membrane by using the turbo-blotting system of BIORAD (Trans-Blot Turbo). The membrane was blocked 45 minutes in 3% BSA-PBS at room temperature on a rolling table. The membrane was then probed with anti-beta actin antibody or the anti EpCAM antibody diluted 1:1000 in 7 ml 2% BSA-PBS and incubated overnight at 4° C. In the first and subsequent washing steps the volume used was 10-15 ml per wash. The membrane was rinsed twice and washed in 3×5 minutes in PBS. The anti-beta actin antibody was detected by using anti rabbit IgG Cy3 conjugate (Sigma Aldrich, cat no. C2306) 1:1000 in 7 ml 2% BSA-PBS for 2 hours at room temperature. The anti EpCAM antibody was detected with streptavidin alexa fluor 488 1:1000 in 7 ml 2% BSA-PBS for 2 hours at room temperature. The membrane was then rinsed once in PBS, washed 4×5 minutes in PBS-tween20 (0.05%), washed 2×5 minutes in PBS, and finally rinsed in PBS. To detect the signal, we used a typhoon scanner (Amersham), which was set for 200 micron resolution and PMT voltage to 500.

### Flow Cytometry

HT29 cells were seeded on anti EpCAM conjugated scaffolds (1000 cells/filter) and cultured for 10 days in a 96-well culture plate before harvesting the cells from the scaffolds by using trypsin. Cells from one scaffold were then incubated with 10 μg/ml of the anti EpCAM antibody in PBS for 30 minutes at room temperature, washed once in PBS and centrifuged at 400 g for 5 minutes. The anti EpCAM antibody was detected by using 1:100 diluted streptavidin alexa fluor 488 (Thermofisher, cat no. S11223) for 25 minutes at room temperature. Washing was repeated, the cells were resuspended in 300 μL PBS, and the flow cytometry was performed on a SONY SH800 Cell Sorter. Non-stained cells detached from a separate scaffold was used as a negative control.

### Culture After Capture

Three filtrations were performed; two non-spiked samples (200 HT29 cells) and one spiked sample (200 HT29 in 4 ml blood). After filtration, the filters were gently rinsed in PBS and transferred to complete growth medium and cultured in a 24-well TC plate (SARSTEDT, cat no. 83.3922) at 37° C., 95% humidity and 5% CO₂, this was considered as day 0.

The next day, the filters were gently rinsed in DPBS and transferred to new wells holding fresh medium. The transfer step was repeated on day 5 and 12. At day 21, the culture was ended, the filters were rinsed in DPBS and fixed in 4% formaldehyde for 10 minutes. Finally, the filters were stained for CD45 and pan-cytokeratin and evaluated by fluorescence microscopy.

### Example 7—Alternative Filter Designs

### Aim

To test Alternative filter designs to increase the capture efficiency and to decrease the filter production time.

### Method

The filter was produced by the method described in Example 1 and 2 but using a different filter design, from here on referred to as the “spiral design” (see FIG. 7). The capture efficiency of the spiral design was evaluated as described in Example 5.

### Results

The spiral design displayed a 51.2% capture, which is a slight improvement compared to 47% of the previous design (Example 1, FIG. 1). The capture efficiency could be further increased to 68.2% by increasing the filter diameter from 12 to 24 mm (FIG. 8). Using the spiral design, the filter production time is decreased by a factor 3.

### Conclusion

By improving the filter design (spiral design) the time it takes to produce the filter could be reduced while maintaining the capture efficiency. Compared to e.g. Takayuki U et al discussed above, in here is demonstrated an ultrathin (10-20 pm) filter for capture and on site culture.

### Overall Conclusion on Results in Examples 1-7

In conclusion, a scaffold/filter (1) for capturing and culturing CTCs, where cancer cell clusters can be formed within two weeks, has been developed. A key element of the capture and culture device is that the CTCs are dispersed on the filter as single cells or clusters of few cells, from where the cells grow to clusters. The expanded clusters thus represent one or a few clones of the tumor, hereby facilitating the identification of important clonal subsets of the cancer. These cluster subsets of patient CTCs might hold valuable information such as drug resistance and DNA mutations, and will thus bring the clinicians closer towards personalized medicine management. Further, the apparent 2D structure of the filter and its transparent nature, makes it possible to directly analyze the filter under the microscope to e.g. visualize cultured CTCs present on the filter.

1. A filter comprising:
a frame layer of fibers; and
a filtering layer of fibers, comprising cell capturing moieties
wherein the filtering layer has a thickness in the range 1-100 μm and has a coiled or nonlinear form;
wherein the frame layer has a thickness in the range 10-100 μm; and
wherein the frame layer (4) has a linear form with a spacing around of about 100-500 μm.

2-31. (canceled)

32. The filter according to claim 1, wherein the filter is transparent, and configured for light microscopy.

33. The filter according to claim 1, wherein the fibers are fabricated by melt electrospinning writing (MEW).

34. The filter according to claim 1, wherein
the frame layer has a thickness in the range 40-100 μm, and
the filtering layer has a thickness in the range 2-20 μm.

35. The filter according to claim 1, wherein the filtering layer has an average maximum pore axis in the range 8-50 μm.

36. The filter according to claim 1, wherein the filtering layer has an average fiber diameter in the range 1-6 μm.

37. The filter according to claim 1, wherein the fibers of the filtering layer have 10-100 coils per mm.

38. The filter according to claim 1, wherein the filtering layer has a porosity in the range 30-80%.

39. The filter according to claim 1, wherein the fibers of the filtering layer have a coiled form and the fibers of the frame layer have a linear form.

40. The filter according to claim 1, wherein the filter is configured for capturing circulating tumor cells (CTCs).

41. The filter according to claim 1, wherein the fibers comprise a material selected from the group consisting of Polycaprolactone (PCL), Polylactic acid, Poly(lactide-co-glycolide), Poly(methyl methacrylate), Polypropylene, Polyethylene, Poly(caprolactone-block-ethylene glycol) and Polyurethane.

42. The filter according to claim 1, wherein the filter is configured for growing cells thereon.

43. The filter according to claim 1, wherein said filter is integrated in a device selected from the group consisting of a filtering device, a cell culturing device and a microscope.

44. A method for capturing cells, the method comprising:
a) providing a filter according to claim 1; and
b) passing a sample comprising cells through the filter, thereby capturing the cells bound by the cell capturing moieties of the filter.

45. The method according to claim 44, wherein the cells are circulating tumor cells (CTCs).

46. The method according to claim 44, wherein the sample is passed through the filter at a rate of 0.2-4 ml/hour.

47. The method according to claim 44, wherein the sample volume is in the range 0.1-20 ml.

48. A method for capturing and culturing cells, the method comprising
a) performing the method according to claim 44;
b) transferring the filter to a culturing medium for the cells; and
c) culturing the cells on the filter.

49. The method according to claim 48, wherein the cells are CTC's.

50. The method according to claim 48, wherein in step c) the cells are cultured for at least 1 day.
